(19) United States
(12) Patent Application Publication
(10) Pub. No.: US 2022/0215914 A1
(43) Pub. Date: Jul. 7, 2022

(54) Method Of Implementing a Decentralized User-Extensible System for Storing and Managing Unified Medical Files

(71) Applicant: Samir Issa, DUBAI (AE)

(72) Inventor: Samir Issa, DUBAI (AE)

(21) Appl. No.: 17/571,444

(22) Filed: Jan. 7, 2022

Related U.S. Application Data
(60) Provisional application No. 63/134,718, filed on Jan. 7, 2021.

(51) Int. Cl. G16H 10/60; G16H 15/00

(52) U.S. Cl. CPC G16H 10/60; G16H 15/00

(57) ABSTRACT

A method of implementing a decentralized user-extensible system for storing and managing unified medical files resolves fragmentation of medical records, avoids the creation of uncontrolled centralized healthcare data repositories, and empowers patients to control and manage the read-write access to their clinical information. Furthermore, the method creates an open, user-extensible, and shared data model at the level of user interface by implementing unified medical file (UMF) web controls that have several user interfaces specifying attributes and can at the same time reference international code sets. The particular difference of the method from all others is that the common data model is implemented at the user interface level, and that semantic unity is achieved at the front end initially and at the back-end naturally afterwards.

The current application claims a priority to the U.S. Provisional Patent application Ser. No. 63/134,718 filed on Jan. 7, 2021.

### FIELD OF THE INVENTION

The present invention relates generally to electronically storing and retrieving medical information. More particularly, the present invention relates to a method for solving medical record fragmentation by enforcing user interface semantic unity in a decentralized, user-extensible data model implemented through user-configurable dynamic web controls and web form templates.

### BACKGROUND OF THE INVENTION

The problem of fragmentation and dispersion of a patient's medical information is a well-known and challenging one. Even with the existence of electronic digital filing systems, document databases, and cloud computing, no software solution has been able so far to assemble and compile the entirety of clinical information of a person in a single repository; this is because of the distributed nature of medical service provision.

Medical services are delivered by various providers. Each provider implements their own business rules to collect, store, and analyze medical information. A uniform solution imposed by authority may achieve this end, however it is not an acceptable approach. An imposed solution alienates healthcare providers who become deprived of freedom in exercising their knowledge to collect and analyze information and provide care.

Enforcing a common data model at the level of data repository/backend is an important step towards achieving semantic unity in the namespace of the application domain; however, adopting a common data model based solely on international codes is not enough because it restricts the data model vocabulary to technical medical terms; this prevents users of the model of using data input forms that supports the business rules of their practice even though it achieves back-end semantic unity. An open and user-extensible, yet finite common data model at the level of user interface, is the ideal solution because it enables healthcare providers to add any user-required terms to the common model, to apply their own business rules, and to share subsequently a common data reporting platform between the users of such system.

The present invention resolves fragmentation of medical records, avoids the creation of uncontrolled centralized healthcare data repositories, and empowers patients to control and manage the read-write access to their clinical information. Furthermore, the present invention creates an open, user-extensible, and shared data model at the level of user interface by implementing unified medical file (UMF) web controls that have several user interface specifying attributes and can at the same time reference international code sets. The particular difference of this invention from all others is that the common data model is implemented at the user interface level, and that semantic unity is achieved at the front end initially and at the back-end naturally afterwards.

Additionally, the present invention enables creation of an unlimited collection of web reports that contain new information compiled from data collected through an unlimited quantity of UMF webforms sourced from different providers across many locations.

The present invention is designed in consideration of several premises:

Premise One: A unified medical file is important for timely and proper delivery of health care.

Premise Two: A physically unified medical file is superior to a virtually unified medical file.

Premise Three: A physically unified medical file is useful only if the common data model i.e., semantic unity is implemented across all elements of the file structure.

Premise Four: Semantic unity across all elements of the file structure is only possible if it is implemented at the level of the user interface webforms.

Premise Five: A user interface implementation of semantic unity is only possible if all elements of the user interface i.e., all controls of all webforms are digitally encoded.

Premise Six: Encoding of all elements of the user interface webforms can happen: 1) if uniform webforms are used across the boards, or 2) if encoded controls can be combined as needed to create user interface webforms.

For an electronic medical record (EMR) system to qualify for being a unified medical file, the EMR should be physically unified and not virtually unified—physically unified meaning all portions of the EMR being stored in the same data repository, on the same physical device; while a virtually unified file may be spread across multiple data repositories and/or storage devices. It is important to recognize this distinction because a virtually unified medical file lacks the ability to be queried for a specified string for a specified patient; this is because it is realistically impossible to query all healthcare provider systems in search for the target string. The only possible solution to this search is a physically unified medical file; a physically unified medical file can be implemented in only one of two socially-acceptable ways:
The authority controls the centralized storage of clinical information.
The patient himself controls his own clinical information.

A physically unified medical file is not meaningful and useful if semantic unity and a common data model are not supported at the level of the user interface. Supporting semantic unity and a common data model at the back-end only permits searching for a ‘literal’ target string across all user input but does not ensure the finding of all matching ‘concept’ hits behind the string since users using different user interfaces can use different ‘literal strings’ to describe/name one ‘concept’ if semantic unity and common data model are not supported at the level of the user interface.

Example: if we are searching for a clinical finding of ‘enlarged liver’, this clinical finding could be labeled or entered by three users in three different systems as ‘Hepatomegaly’, ‘Hepatomegalie’, or ‘Liver enlargement’. If our invention is being used by the three users in three different physical locations using different input forms, the three concepts will be recognized and stored by the system as a single concept and most probably will be encoded by a SNOMED international code value of 275296001 in a physically unified file. However, if our system is not used, to find the target SNOMED Code for a specified patient, the system should query all physical components of the system that could have possibly stored the target code.

Unlike any other existing Electronic Medical Record software, the present invention implements web controls that enforce semantic unity and create a common data model at the level of the user interface. The web controls of the present invention can be combined as needed by users, as many variable webforms as desired to create a fully operational user interface:

1. The web controls of present invention constitute a finite, re-usable, and user-extensible collection of web controls that implement their attributes independently from their containing web form.

2. The web controls of present invention are dynamically created in runtime and are independent from a matching back-end data definition.

3. The web controls of present invention are either pre-encoded, or runtime-encoded by an internationally recognized id-code value; this value types the web control with a known medical concept.

4. The web controls of present invention can trigger code execution at the client or server sides; this permits the control to support and behave in a pre-programmed manner in response to user input.

The system of the present invention creates, at the level of the user interface, indivisible atomic data items, or ‘ditems’ that are enriched by a collection of descriptive metadata attributes; these ‘ditems’ are combined to create application webforms in the present invention.

Semantic unity implies that several literal strings (even in one or more languages) are referred to as a single human concept. The web controls of present invention are enriched with a collection of metadata attributes that create the semantic unity between the said web controls;

Certain attributes of the ‘ditem’ represent international codes in an internationally recognized coding system such as SNOMED, ICD10, CPT, LOINC. This enables the user interface to directly encode user input with internationally encoded concepts.

As such, the web controls of present invention seamlessly implement multi-lingual user interface for system labels; when the international code set supports a second language, cross-user/cross platform/cross-language data input and data reporting becomes feasible.

Further, distinctly from any other application, the entirety of an EMR file of a person can be placed on the person's mobile device in their default selected language, and the file can be translated seamlessly to another language supported by the system.

Further, the present invention further enables automatic coupling of clinical activities with financial and store transactions, ensuring proper financial management in healthcare facility.

Additional advantages of the invention will be set forth in part in the description which follows, and in part will be obvious from the description, or may be learned by practice of the invention. Additional advantages of the invention may be realized and attained by means of the instrumentalities and combinations particularly pointed out in the detailed description of the invention section. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

FIG. 1 is an overview of various aspects of the present invention.

FIG. 2 is an overview of various databases utilized in the present invention.

FIG. 3 is an overview of some method steps in the present invention.

FIG. 4 is an overview of an exemplary internal structure of the present invention.

FIG. 5 is a stepwise flow diagram illustrating the general method steps of the present invention.

FIG. 6 is a stepwise flow diagram illustrating steps for creating, storing, and retrieving webform templates and patient data items in the method of the present invention.

FIG. 7 is a stepwise flow diagram illustrating steps for implementing user permissions in the method of the present invention.

FIG. 8 is a stepwise flow diagram illustrating steps for producing a runtime-contextualized filled webform in the method of the present invention.

FIG. 9 is a stepwise flow diagram illustrating steps for managing patient clinical data in the method of the present invention.

FIG. 10 is a stepwise flow diagram illustrating additional steps for managing patient clinical data in the method of the present invention.

FIG. 11 is a stepwise flow diagram illustrating steps for implementing activity templates in the method of the present invention.

### DETAIL DESCRIPTION OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising”, when used herein, specify the presence of stated features, steps, operations, elements, various embodiments, and/or components, but do not preclude the presence or addition of one or more other features, steps, operations, elements, various embodiments, components, and/or groups thereof.

Unless otherwise defined, all terms (including technical and scientific terms) used herein have the same meaning as commonly understood by one having ordinary skill in the art to which this invention belongs. It will be further understood that terms, such as those used in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques, embodiments and/or steps are disclosed. Each of these has individual benefit and each can also be used in conjunction with one or more, or in some cases all, of the other disclosed techniques, embodiments and/or steps. Accordingly, for the sake of clarity, the present disclosure will refrain from repeating every possible combination of the individual steps, techniques or embodiments in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident however, to one skilled in the art, that the present invention may be practiced with or without these details or with other similar or otherwise suitable details.

The present disclosure is to be considered as an exemplification of the invention, and it is not intended to limit the invention to the specific embodiments illustrated by the figures or descriptions.

In the present disclosure, the term “end user” may refer to a computer user who does not have any programming or coding knowledge. A “software application” or “application” is a compiled code that executes specific functions built to meet specific end user requirements and is immediately available, without further coding required from end users. A “software engine” or “engine” is a compiled code that executes generic computer functions that are built to meet generic end user or environment requirements, and is immediately available, without further coding required from end users. A “programming method” or “programming application” is a software application that helps software programmers to design and encode a software application.

The present invention relates to a software engine that may employ several software modules that enable an end user to design, create, and use an unlimited number of different types of forms, reports, and queries over an electronic network connection such as, but not limited to, a local area connection, a wide area network, the Internet (which may be referenced to in some instances as “the web” or variations thereof), or any other suitable type of network connection. The method steps claimed do not necessarily directly reflect any specific coding or other implementation details of the present invention, rather the general logical process of facilitating the operation of the software engine. Various embodiments of the present invention may comprise and implement various software modules as desired and deemed necessary for real-world implementation; however, in the current disclosure, it is generally considered of not great importance which particular software module performs what action, so long as the action is enabled and carried out by some processing component of the present invention capable of adequately doing so.

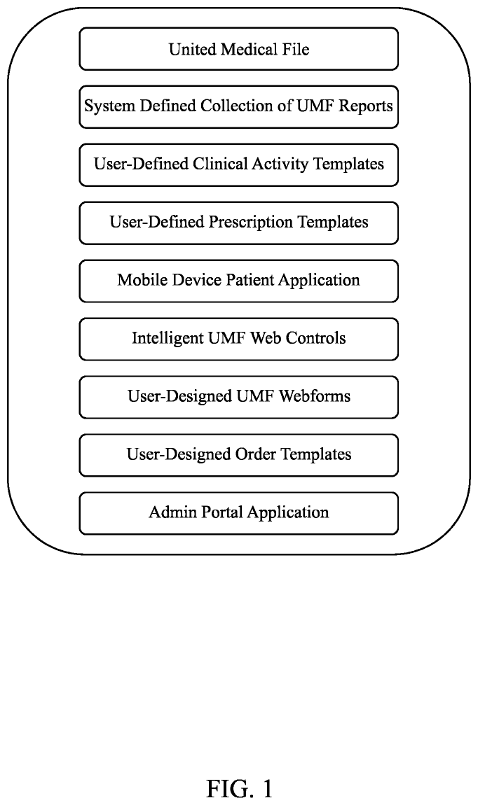

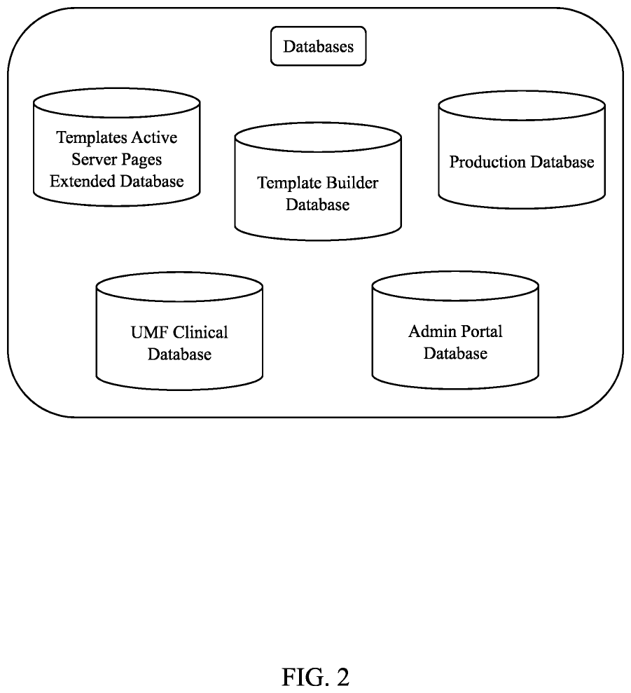

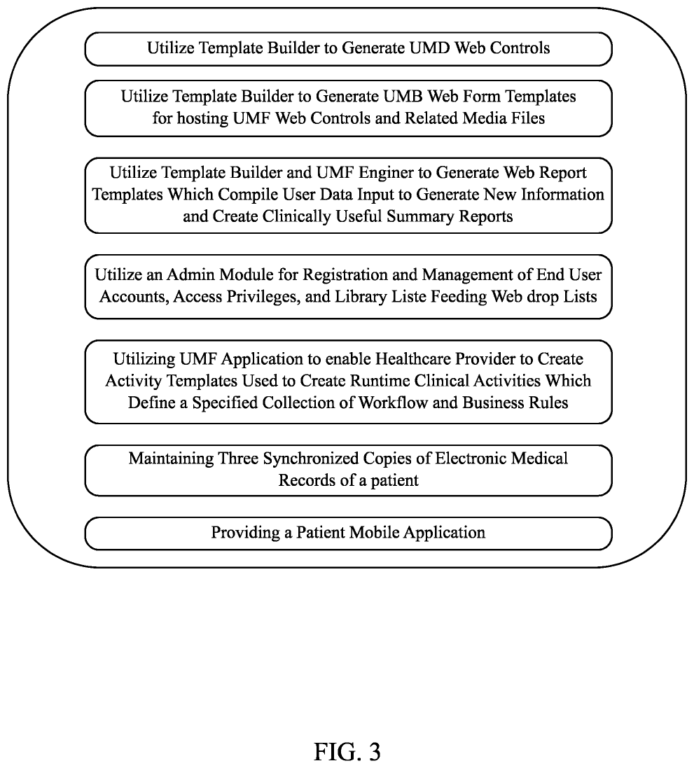

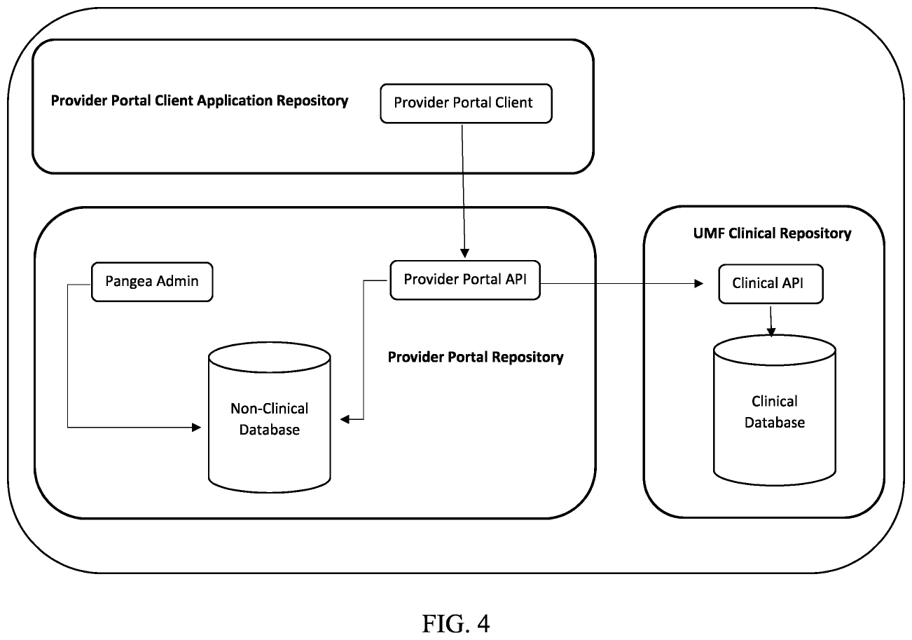

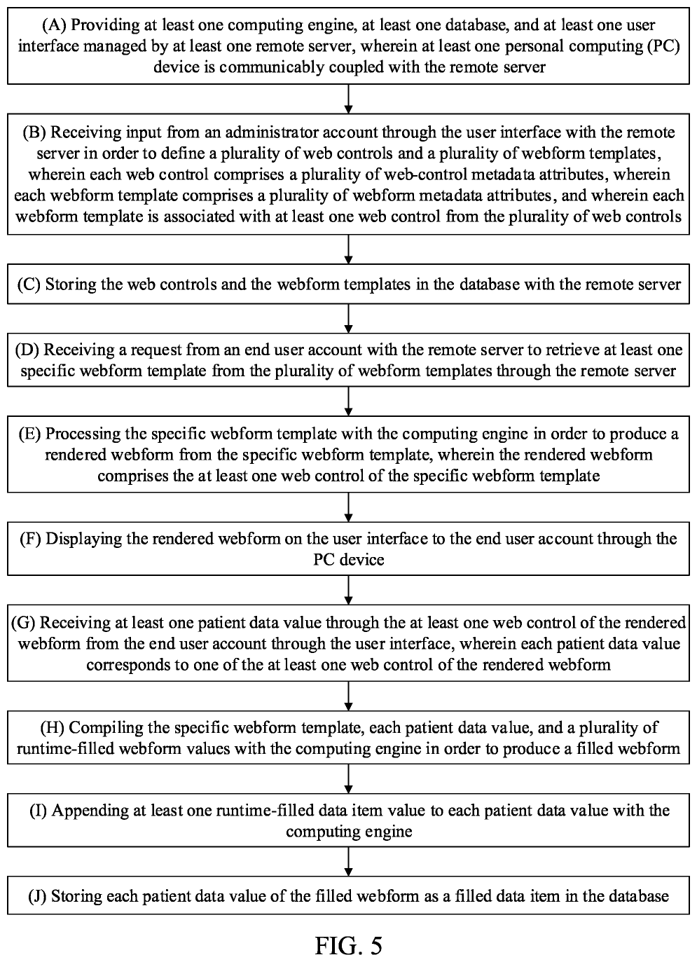

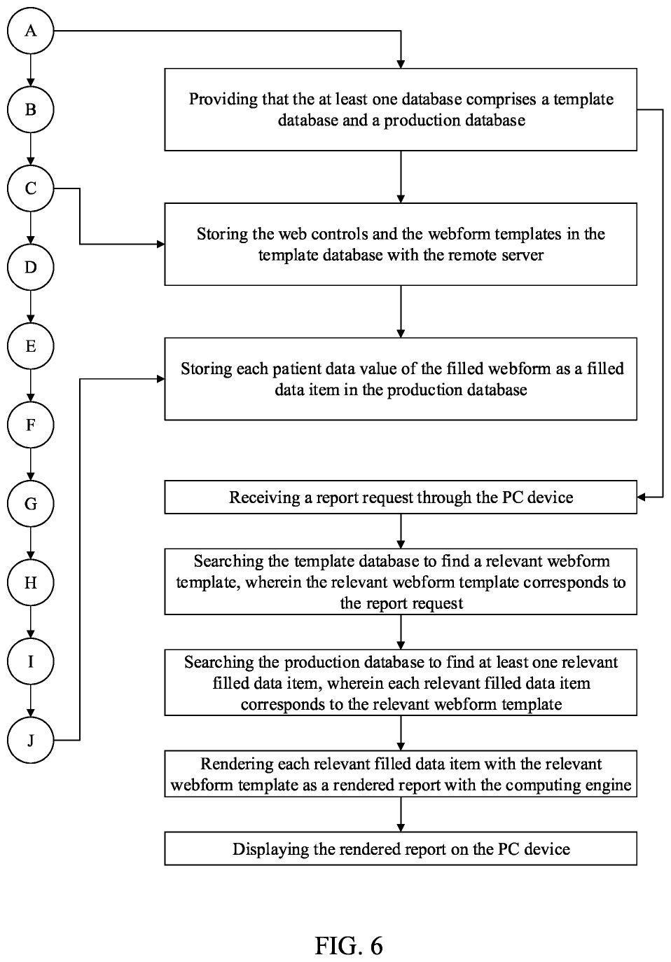

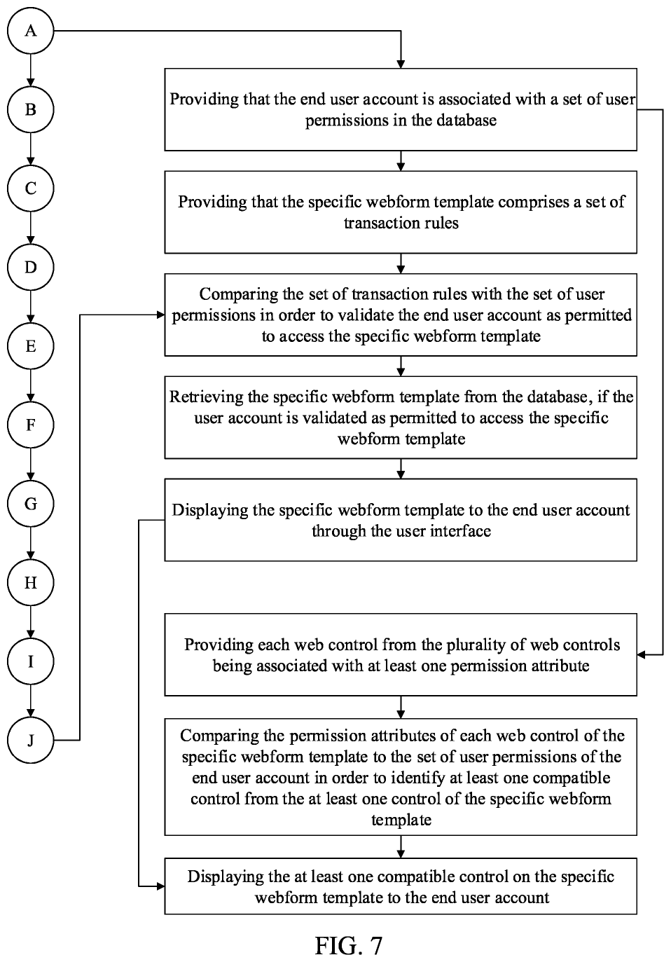

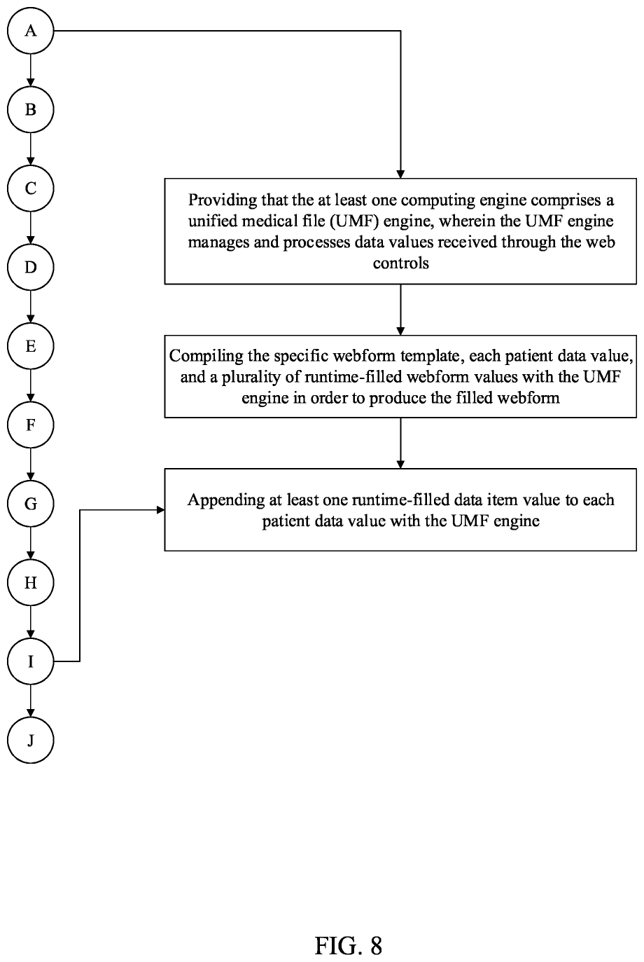

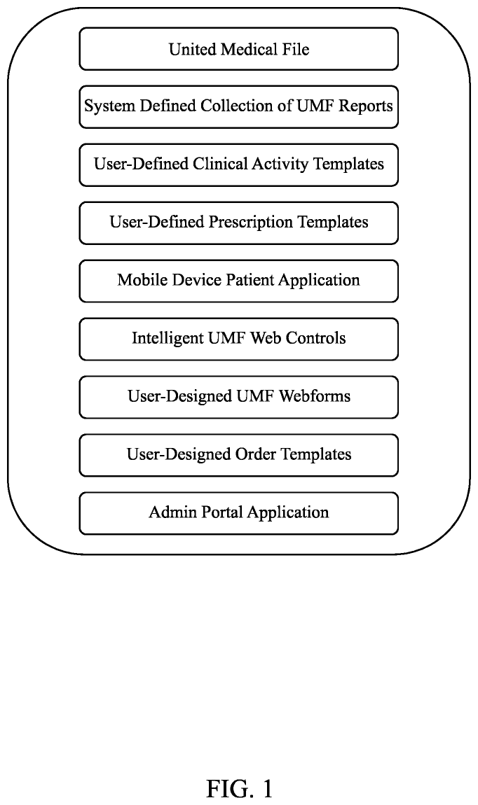

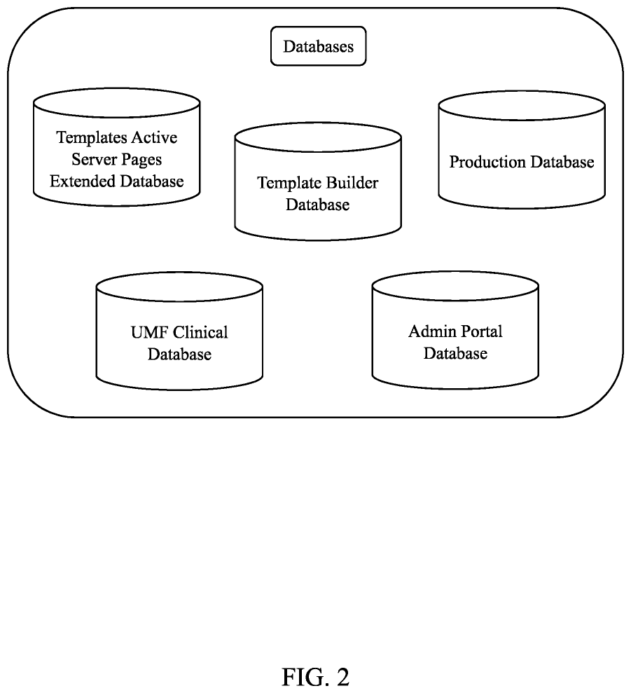

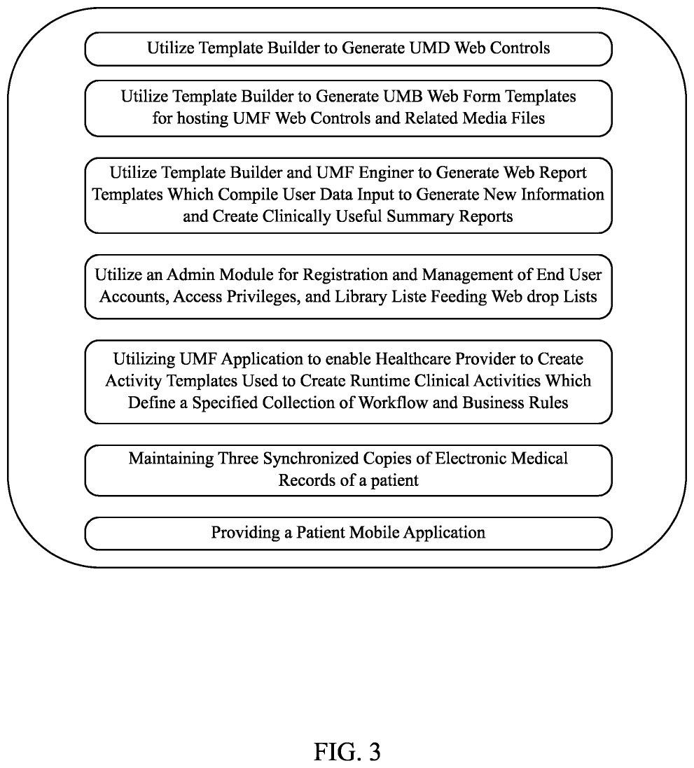

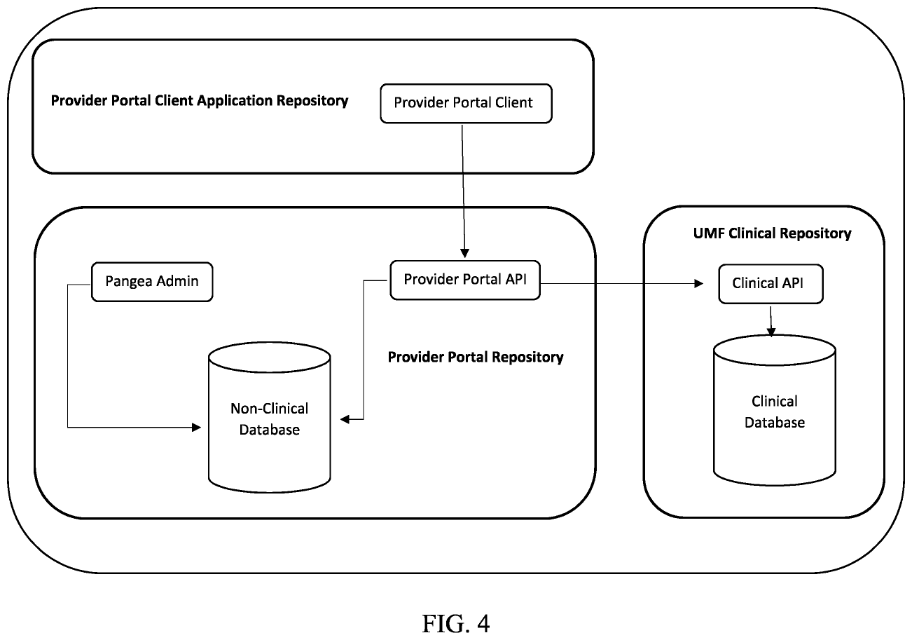

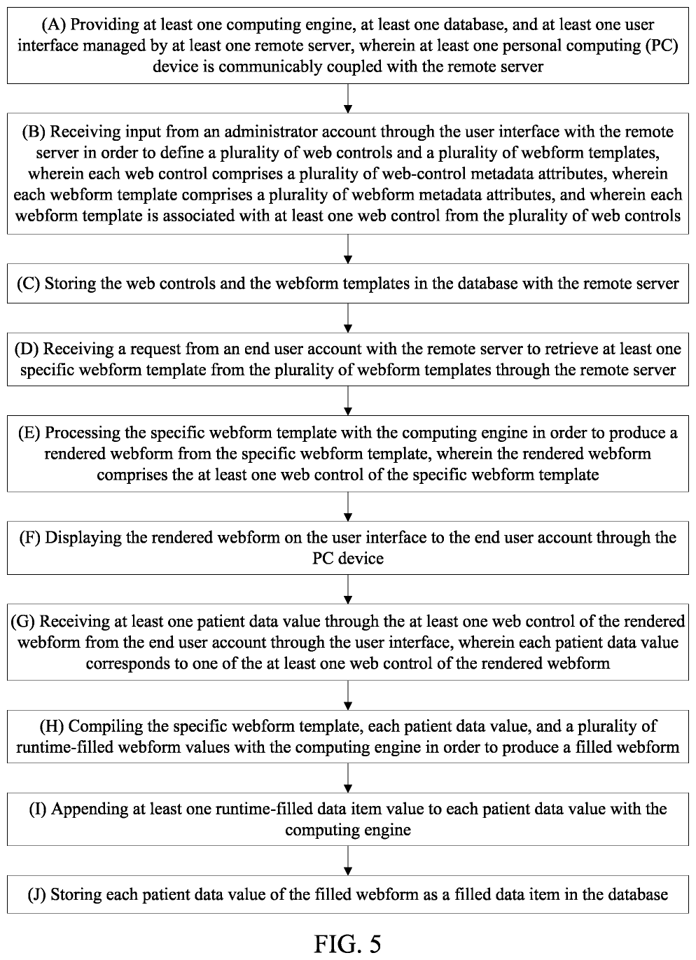

In various embodiments, the present invention may comprise multiple databases, of any suitable type, structure, architecture, configuration, or other relevant attribute. In an exemplary preferred embodiment, the present invention comprises five database components. The database components for the exemplary preferred embodiment include a template builder database, a template Active Server Pages Extended (‘ASPX’) database, a production database, a UMF clinical database, and an admin portal database. A general overview of the present invention is shown in FIGS. 1-4.

Further, the exemplary preferred embodiment comprises a plurality of software application components, or software modules; in some embodiments, the terms may be used interchangeably as appropriate. More particularly, the plurality of software application components in the exemplary preferred embodiment comprises six application components:

The first application component for the exemplary preferred embodiment comprises a template builder, which is used to build templates of web controls and webforms. The template builder depends on the admin portal application to populate certain values in the templates. The template builder interacts with two databases, the template ASPX database and the template builder database.

The second application component for the exemplary preferred embodiment comprises a Pangea data engine which reads and writes to the database containing the clinical information such as the production database.

The third application component for the exemplary preferred embodiment comprises an admin portal application which reads and writes to the admin portal database.

The fourth application component for the exemplary preferred embodiment comprises a UMF clinical application which reads and writes to the UMF clinical database. The UMF clinical application depends on the Pangea data engine to read and write to the production database.

The fifth application component is a patient mobile application which reads and writes to the UMF clinical application. Lastly, the sixth application is a provider mobile application which reads and writes to the UMF clinical application.

In a general overview of the present invention, a template builder module or application allows an end user to build computer-readable templates within a digital environment for any desired purpose. The templates are extremely versatile and may be built with any number or structure of data inputs, web controls, rules, user restrictions, executable instructions, or other attributes. Once built, a template is stored in a template database, from which it can be retrieved for use. An end user may retrieve the template by requesting it through a graphical user interface (GUI). The template is rendered as a web form on the GUI, and the end user inputs values and otherwise interacts with the web form. The web form receives runtime data when the end user saves or submits the web form, and the newly acquired data is bundled and stored in a production database.

In the preferred embodiment of the present invention, the template builder lets its users create web form templates that host UMF web controls and runtime uploaded related media files. The user-designed UMF web forms comprise a collection of UMF web controls, have a rich collection of attributes to classify and control privileges of management, filtration, and access of the web form, and have a related specified business logic that is activated by certain runtime values in the web form attributes or the values of the contained web controls.

The runtime UMF web forms are not a simple container of web controls, rather, a web form template, like the web controls of UMF, comprises a collection of metadata attributes describing the shared context of occurrence of the component controls and the context of occurrence of their ‘Value’ attribute update. The collection of attributes of the webform specifies logical attributes, presentation attributes, and programmatic values that control the filtration, appearance, and processing functions of the webform during runtime. The attributes include but are not limited to ID of the web form, title of the webform, patient ID, author ID, generating work group, target work group, owner healthcare facility ID, date and time of creation, status, and parent clinical activity ID. Further, the web form preferably displays a QR code that may be captured and used during runtime to upload different media files attached to the web form.

As a general overview of the present invention, the present invention is a data value-centered programming method and software engine for capturing, storing, organizing and reporting data in an organizational environment through user created web based templates and web form controls that enables end users to design, create and use an unlimited number of different types of forms, reports, and queries over a computer network. The present invention is a data capture, storage and retrieval software system that handles data generated during business domain transactions.

More particularly, the present invention is a method of implementing a decentralized, user-extensible system for storing and managing unified medical files (UMFs) by executing computer-executable instructions stored on a non-transitory computer-readable medium. Furthermore, the present invention creates an open and shared data model by implementing UMF web controls that reference international code sets and that can support a specified logic execution in relation to the value entered in the control.

User accounts are able to add and read information to and from a data repository that comprises web control values. The present invention comprises a UMF engine that analyzes and compiles a data value when the data value is entered in a web control. The UMF engine sorts, classifies, and understands the data value inputs.

Furthermore, the present invention provides an unlimited collection of webforms and web reports. A specified collection of UMF controls is grouped under a web form template or a web report template. The UMF web controls are generated via a template builder application. A user interface permits a user account to design templates of UMF web controls and then to group them in different structural arrangements to create templates of UMF web forms;

During runtime, webform web controls gain additional runtime metadata values and capture user input values as well. A plurality of basic and gained metadata values and the web controls' values describe the context of occurrence of the value(s) of the webform in runtime.

The plurality of web controls capture user-entered values during business transactions in runtime. The UMF web controls are able then to own a collection of metadata attributes, implement a specified logic, and assign an initial data value to those metadata attributes.

The intelligent UMF web control is designed by a user account in a pre-made web control template that assigns an initial value to some metadata attributes describing the web control ‘Value’ attribute whose string value will be later captured in runtime when entered by an end user account along with the context of occurrence of the value represented by the plurality of metadata attribute values of the web control and its container webform. Furthermore, certain metadata attributes can reference values from any required international code set. Subsequently, the metadata values control the behavior, appearance, and business logic of the controls.

In runtime, the initial web control metadata value assignment may comprise, but is not limited to:

1. A Global Unique Identifier (GUID) value (this happens when the web control becomes instantiated as a component of an instantiated webform that itself has its own runtime GUID) and only by the system for the present invention;

2. A unique identifying name in the UMF namespace;

3. A base data type (integer, string, Boolean, float, date, etc.) accepted by the web control;

4. A the string ‘Value’ attribute is normally empty and ready to receive an end-user account input during runtime, example digit ‘5’;

5. The metadata attribute ‘Value Qualifier’ string qualifies the ‘Value’ and is updated by an end user account input in runtime from a drop list that is itself set during design time, example: (milligram, degree centigrade, or millimeter of mercury, etc.) qualifying the digit ‘5’ entered as a string by the end user; this leads to the system acquiring a value of 5 of data type integer and recognizing it as 5 milligrams when the qualifier is assigned as such.

Web report templates are composed of collections of intelligent web controls; they are instantiated during runtime and collect production data of their web controls from the database according to the report template-specified filters and display it in the format and order specified in the web report template.

The collection of the plurality of UMF web controls and the plurality of therein associated metadata values control access and programmatic behavior of the controls, the forms, and the application at further stages of the method for the present invention. Additionally, processing the UMF web controls' data provides new information to better render, present, and understand a patient's clinical condition. Furthermore, UMF information is displayed on a single web page containing web links to deeper details that are available for access as needed.

Furthermore, the initial value assignment, in the web control template state, may also comprise, but is not limited to:

1. Optionally assigning a dictionary name (to reference an internationally recognized code set such as Health Level 7 HL7 Message, or FHIR resource [HL7 organization], Systemized Nomenclature of Medical Clinical Terms [SNOMED], Current Procedural Terminology [CPT], International Classification of Diseases [ICD], etc.),

2. Optionally assign a dictionary chapter (referencing a chapter/sub class of the internationally recognized code set such as ‘Order’ of HL7, ‘Clinical Finding’ of SNOMED, etc.),

3. Optionally assign an Item ID (referencing the specific ID value of the item in the internationally recognized code set such as order of HL7, 301113001 of SNOMED for SNOMED clinical finding, heart rate, etc.). Alternately, this value of Item ID can be updated in runtime by user interface logic parsing of a user-selected value from a drop list element to extract a specific ID of the international code of the dictionary specified in the web control.

4. An ‘Annotation’ attribute updated (if needed by the end user account during runtime to add comments on the element's value, and owner webform ID (represent the ID of the container webform).

Furthermore, the initial value assignment may also comprise but is not limited to optionally assign a set of business logic execution conditions and code, for example: (if ‘Value’ is more than 5), that can trigger the execution of a specified server logic that can propagate through the whole system.

The template builder and the UMF engine create web reports that compile user data input in UMF web controls to create a set of standardized, built-in, clinical reports in relation to a provider, a facility, or an interval, or another relevant attribute. Furthermore, the template builder and UMF engine enable user accounts to create, similarly to web controls and webforms, templates of patient-centric web reports whose information is sourced from user accounts input in UMF namespace web controls surviving in a single data repository and updated in the web forms of several healthcare facilities by multiple providers of different user types including doctors, nurses, technicians, etc. This characterizes the system of the present invention from other systems that are facility-centric; facility-centric systems are limited to process the information gathered through the specific data input forms of the facility conforming to its business rules; these forms cannot be shared with other facilities because of the lack of a common data model definition encompassing the individual data elements characterizing the business transactions of each and every single facility.

Only the aforementioned architecture permits the real time compilation of a patient medical file's findings across facilities and across providers. Without sharing the same namespace of data elements and sharing the same production data repository, it is impossible to generate a single real time, patient-centric, unified medical file.

The present invention is disclosed herein as a method of operating a software application that resolves fragmentation of medical records, avoids the creation of uncontrolled, centralized healthcare data repositories, and empowers patients to control and manage the read/write access to their clinical information.

Additionally, the database table definition and the data repository architecture of the present invention enables the creation of an unlimited collection of web reports containing new information compiled from already collected data sourced from different healthcare providers across many locations. The UMF web controls for the present invention provide a common and open/user-extensible data model under one programmatic namespace. Furthermore, the user input forms, and the user output reports share the data of the UMF web controls across the boards and meet the needs of all user account types.

Referring to FIG. 1, in the general method of the present invention, at least one computing engine is provided, along with at least one database and at least one user interface, the foregoing being managed by at least one remote server, which is further provided in the present invention (Step A). Further, at least one personal computing (PC) device is provided and communicably coupled with the remote server. It should be noted that the at least one computing engine, the at least one database, the at least one user interface, the at least one remote server, and the at least one PC device should each be understood to be a generalized term for a group of one or more of the named elements, and in different embodiments, may comprise different quantities of the named elements without limitation, and may further be understood in some cases to technically refer to different instances of the named elements as may be comprised in any given implementation, but may be understood to fulfill the same generalized role across such disparate instances, such that a singular phrasing of an element's name may refer to such instances interchangeably as may be understood in the context in which it occurs without departing from the spirit and scope of the present invention. It may be further understood that, for elements with a prefix of “at least one”, the element may be understood to represent an undefined group consisting of as few as a single constituent member or multiple constituent members without limit as desired in various different embodiments of the present invention. It may further be noted that such groups of “at least one” may in some instances be referred to in a singular tense for the sake of brevity, or in a plural tense where the context is appropriate, without limiting the referenced group to a single constituent member.

In the preferred embodiment, the current invention's software engine utilizes a table definition structure that hosts all end user input and runtime data of the UMF controls in a single table. This architecture renders possible the cross reporting and intelligent analysis of UMF web controls' data from a single data repository; otherwise, and in all currently implemented solutions of electronic medical records, this data resides in disparate tables, in one or more disparate databases, where cross reporting is realistically impossible, and the only way to integrate patient's information for clinical use is by implementing inter-operability protocols such as HL7 messages or FHIR resources exchanged as FHIR-structured JSON documents.

To begin the substance of the method, in the preferred embodiment of the present invention, input is received from an administrator account through the user interface with the remote server in order to define a plurality of web controls and a plurality of webform templates, wherein each web control comprises a plurality of web-control metadata attributes, wherein each webform template comprises a plurality of webform metadata attributes, and wherein each webform template is associated with at least one web control from the plurality of web controls (Step B). In the preferred embodiment, each of the plurality of web controls is associated with a specified medical identifier code. The web controls and the webform templates are then stored in the database with the remote server (Step C).

A request is received from an end user account with the remote server to retrieve at least one specific webform template from the plurality of webform templates through the remote server (Step D). The specific webform template is processed with the computing engine in order to produce a rendered webform from the specific webform template (Step E), wherein the rendered webform comprises, or inherits, the at least one web control of the specific webform template.

The rendered webform is then displayed on the user interface to the end user account through the PC device (Step F). At this point, the rendered webform is in a naïve state, ready to accept user input in order to populate the web controls of the rendered webform with numeric values, lexical words, or any other relevant type of receivable data.

More particularly, at least one patient data value is received through the at least one web control of the rendered webform from the end user account through the user interface, wherein each patient data value corresponds to one of the at least one web control of the rendered webform (Step G).

In the preferred embodiment, the present invention comprises a non-centralized deployment structure where a UMF file exists in at least one, and at most three human-readable but encrypted identical copies. First, a copy on the patient's mobile device. Second and optionally, a copy on the last visited healthcare facility server. Lastly and also optionally, one back up copy on UMF cloud servers.

Furthermore, the method for the present invention comprises two-state description for clinical information of a patient. The first state is a hot state. The hot state is when a patient clinical document or activity is actively in-edit mode while the patient is receiving interactive medical care. The second state is a cold state. The cold state is when a patient clinical document is in locked/signed-off mode after delivering medical care.

The specific webform template, each patient data value, and the plurality of runtime-filled webform values is then compiled with the computing engine in order to produce a filled webform (Step H). At least one runtime-filled data item value is appended to each patient data value with the computing engine (Step I), and each newly runtime-contextualized patient data value of the filled webform is stored as a filled data item in the database.

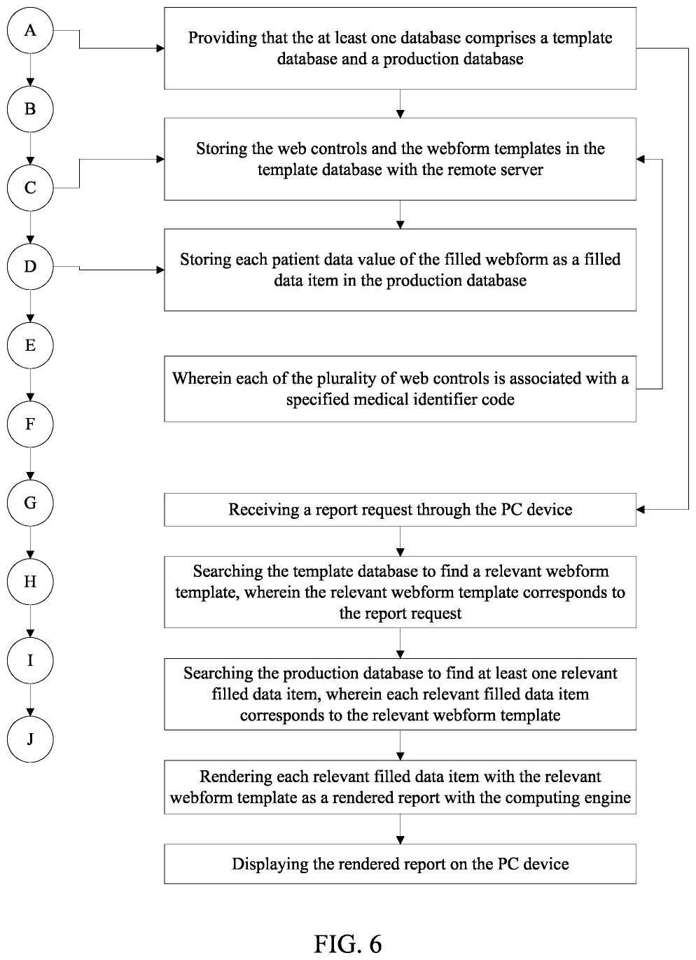

In various embodiments, the at least one database may comprise any suitable quantity and configuration of databases as appropriate for a given embodiment. More particularly, for the purposes of the present disclosure, in the preferred embodiment, the at least one database comprises a template database and a production database. The template database stores definitions for naïve web controls, naïve webform templates, and other relevant template elements. Data is related in the production database in numerous ways, and the database can be queried to show an end user desired data ranges and/or relations. The web controls and the webform templates are stored in the template database with the remote server in Step C. Further, each patient data value of the filled webform is stored as a filled data item in the production database in Step D, as shown in FIG. 6.

Further in the present invention, with the template database and the production database provided, a report request is received through the PC device. The template database is searched to find a relevant webform template, wherein the relevant webform template corresponds to the report request. The production database is searched to find at least one relevant filled data item, wherein each relevant filled data item corresponds to the relevant webform template. Each relevant filled data item is rendered with the relevant webform template as a rendered report with the computing engine, and the rendered report is displayed on the PC device for the end user to view.

In the preferred embodiment of the present invention, the end user account is associated with a set of user permissions in the database. In some embodiments, an administrative software module registers and manages an entity user account access privileges as well as management of library lists feeding the web drop lists.

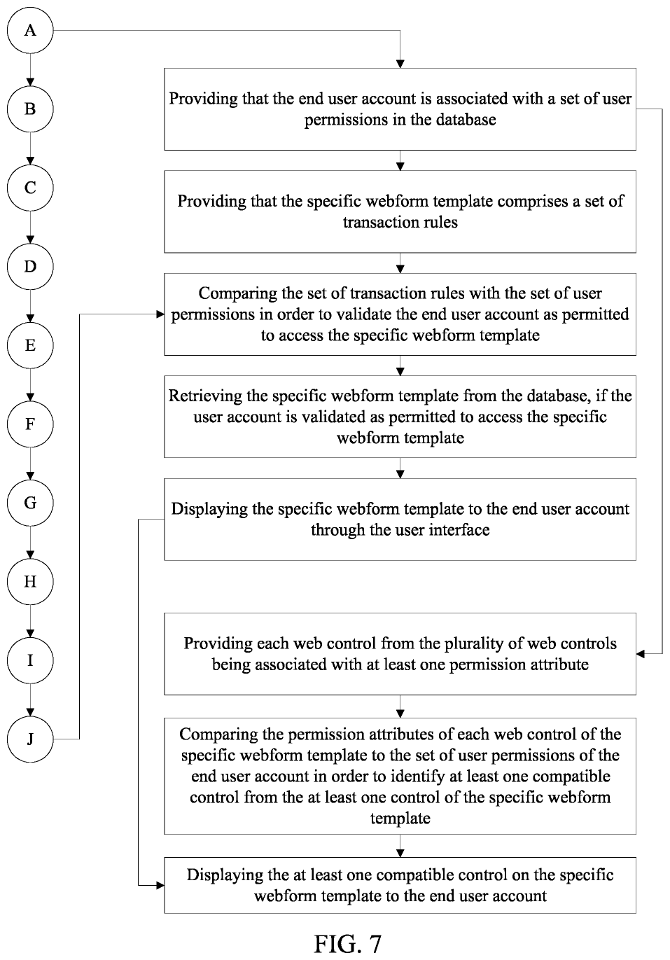

The admin module assigns a set of passkeys to each screen element of the user interface, each passkey represents an access combination to the element; upon login, the system generates a passkey for the user based on his credentials (subscription type, user type, specialty, organization, physical location), and displays only the compatible screen elements. The specific webform template is further comprised as comprising a set of transaction rules, as shown in FIG. 7. The set of transaction rules is compared with the set of user permissions in order to validate the end user account as permitted to access the specific webform template. The specific webform template is retrieved from the database, if the user account is validated as permitted to access the specific webform template, and the specific webform template is then displayed to the end user account through the user interface.

Furthermore, in the preferred embodiment, each web control from the plurality of web controls is associated with at least one permission attribute. The permission attributes of each web control of the specific webform template are compared to the set of user permissions of the end user account in order to identify at least one compatible control from the at least one control of the specific webform template. The compatible controls are then displayed on the specific webform template to the end user account.

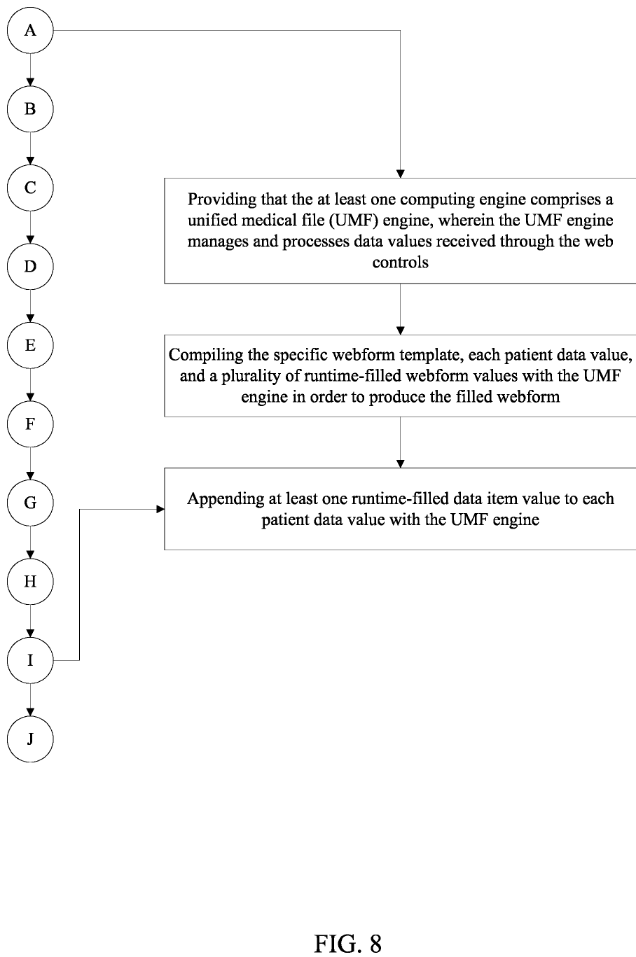

In the preferred embodiment, the at least one computing engine comprises a unified medical file (UMF) engine, wherein the UMF engine manages and processes data values received through the web controls. The specific webform template, each patient data value, and a plurality of runtime-filled webform values are compiled together with the UMF engine in order to product the filled webform in Step G, and at least one runtime-filled data item value is appended to each patient data value with the UMF engine in Step I, as shown in FIG. 8.

In accordance with the desired spirit and scope of the present invention, an end user who is a patient and may be, for example, receiving care at hospital or other medical facility, is provided the convenience of their entire medical file being stored on their personal computing device. More particularly, a patient computing device is provided. The at least one database is further provided as comprising a patient data repository, wherein the patient data repository is stored on the patient computing device. In some embodiments, the patient data repository may simply be a typical internal storage medium belonging to a mobile smartphone device of the patient.

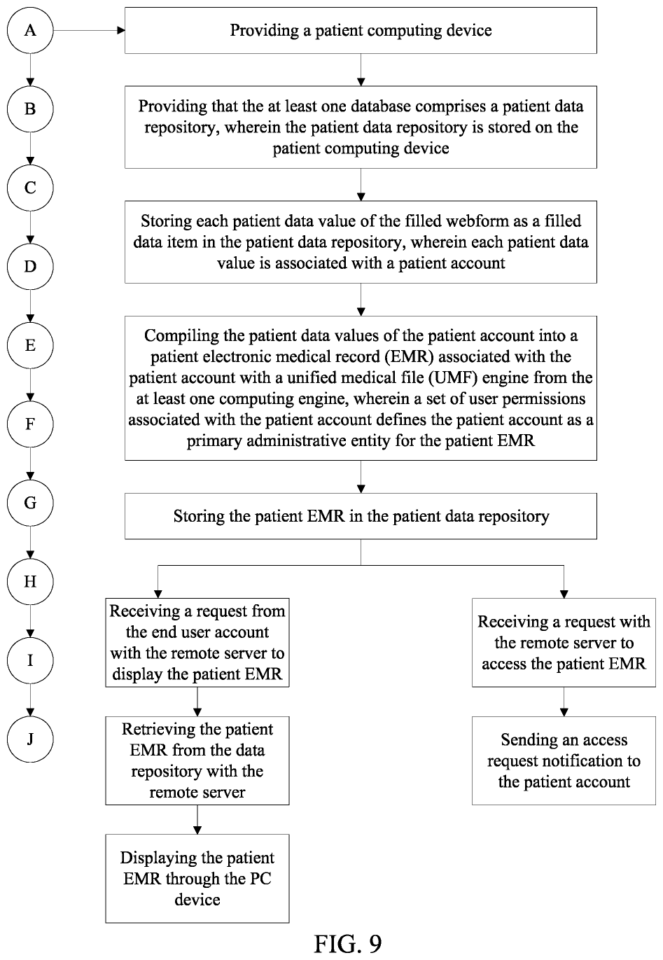

As shown in FIG. 9, each patient data value of the filled webform is stored as a filled data item in the patient data repository, wherein each patient data value is associated with a patient account. The patient data values of the patient account are compiled into a patient electronic medical record (EMR) associated with the patient account with the previously discussed UMF engine, wherein a set of user permissions associated with the patient account defines the patient account as a primary administrative entity for the patient EMR. Finally, the patient EMR is stored in the patient data repository. Thus, the entirety of the patient's medical file is stored conveniently on their mobile device for each of access.

Further, in some embodiments, a request maybe received with the remote server from the end user account to display the patient EMR. For example, the patient's nurse may want to check a particular vital sign, or check the progress of a treatment. The patient EMR is retrieved from the data repository with the remote server, and the patient EMR is displayed through the PC device associated with the end user account.

Further, in the preferred embodiment, the patient may choose to have the system notify them if anyone attempts to access their records. Thus, a request may be received with the remote server to access the patient EMR, and as a result, an access request notification is sent to the patient account.

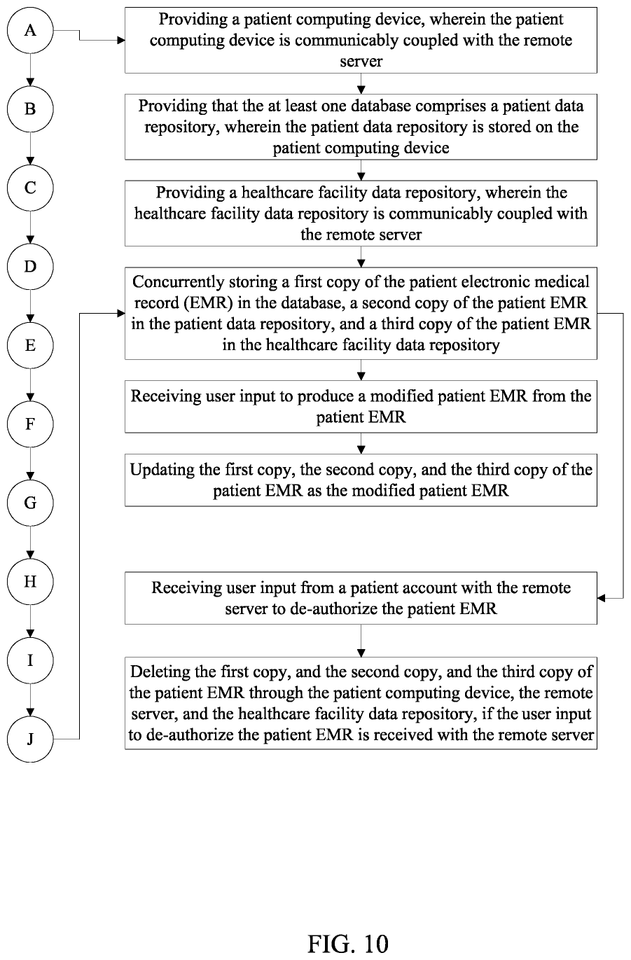

Further, in the preferred embodiment, the patient EMR is stored in multiple locations simultaneously and kept in sync with each other. To this end, referring to FIG. 10, a healthcare facility data repository may further be provided, wherein the healthcare facility data repository is communicably coupled with the remote server, and wherein the patient may be receiving treatment from the healthcare facility, such that the patient's care at the healthcare facility is recorded through the present invention.

A first copy of the patient EMR is stored in the database of the present invention, concurrently with a second copy of the patient EMR being stored in the patient data repository of the patient's mobile device, and a third copy of the patient EMR is further concurrently stored in the healthcare facility data repository. When a change in the patient's medical status or history occurs, undergoing a new treatment for example, or completing a treatment in progress, user input is received through the user interface to produce a modified patient EMR from the patient EMR. This may correspond to a healthcare activity regimen being completed, for example. User input is received through the user interface to produce a modified patient EMR from the patient EMR. Subsequently, the first copy, the second copy, and the third copy of the patient EMR are each updated as the modified patient EMR.

In some embodiments, every time a patient initiates an interaction with a health care provider, a clinical activity is created in the system; the clinical activity is defined by a collection of metadata attributes and a collection of our webforms that define and document the business flow of the activity.

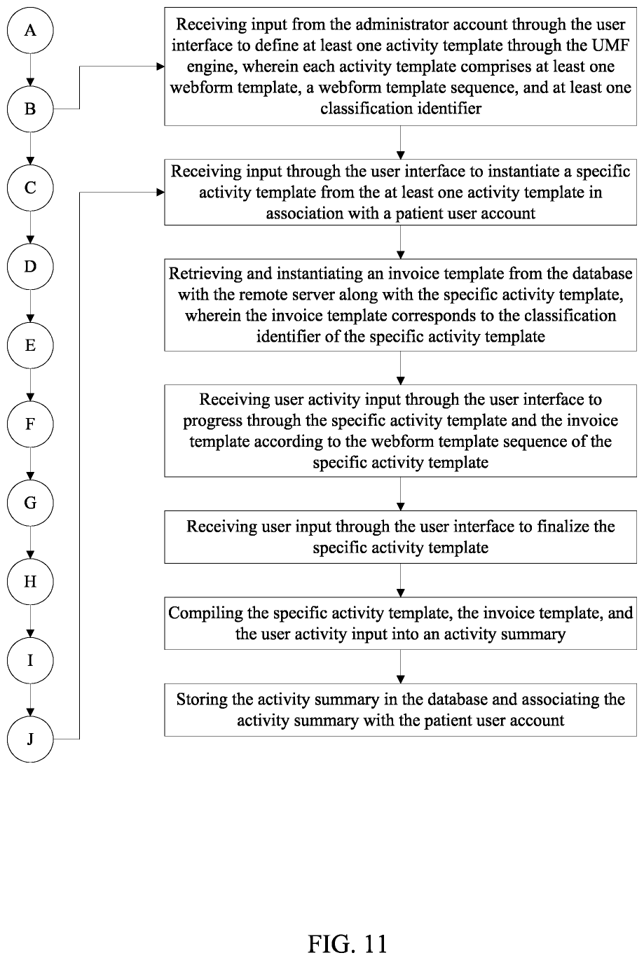

Further, in some embodiments, referring to FIG. 11, every time a patient initiates an interaction with a health care facility, a facility clinical activity called a composite activity is created in the system; the facility clinical activity is defined by a collection of clinical activities that together define a clinical pathway or a clinical protocol.

When a clinical activity, or a composite clinical activity is created, the system automatically retrieves, and instantiates, a corresponding invoice template with a specified price list amount and a related collection of financial transactions and store transactions. This ensures that financial and inventory transactions are consistently generated in the system when activities are executed.

In the preferred embodiment, every time a clinical activity is signed off by an author user account or other relevant account in a healthcare facility, the activity is converted by the UMF engine to an immutable JavaScript Object Notation (JSON)/Extensible Markup Language (XML) document using Structured Query Language (SQL) server data. The immutable JSON/XML document is saved with its peers in a folder on the UMF server that can be replicated on a patient's mobile device.

In the hot state i.e., unsigned, a patient's clinical information is managed entirely on a relational database where all health care providers interact with the documents using UMF forms and data engine webforms; business rules are implemented in the application logic and in the user-designed web controls and web forms.

Further, in accordance with the spirit of the present invention, the patient of the patient account has full control over their medical information. The patient can restrict access to the patient EMR to whoever they want, or the patient can delete their data altogether. To this end, user input may be received from the patient account with the remote server to de-authorize the patient EMR. Alternatively, any other suitable technically different means may be employed in the present invention for the patient to remove their medical data from the system of the present invention. If the de-authorization input is received, the first copy, the second copy, and the third copy of the patient EMR are deleted through and from the patient computing device, the remote server, and the healthcare facility data repository, respectively.

Additionally, the preferred embodiment of the present invention further enables the use of clinical activities with financial and store transactions and the like to ensure proper financial management in a healthcare facility.

To this end, input is received from the administrator account through the user interface to define at least one activity template through the UMF engine, wherein each activity template comprises at least one webform template, a webform template sequence, and at least one classification identifier. Additional input is further received through the user interface to instantiate a specific activity template from the at least one activity template in association with the patient user account.

In invoice template is then retrieved from the database and instantiated with the remote server along with the specific activity template through the user interface, wherein the invoice template corresponds to the classification identifier of the specific activity template.

Then, user activity input is further received through the user interface to progress through the specific activity template and the invoice template according to the webform template sequence of the specific activity template. User input is then received through the user interface to finalize the specific activity template. The specific activity template is then compiled through the remote server along with the invoice template and the user activity input into an activity summary, and the activity summary is stored in the database and associated with the patient user account.

Although the invention has been explained in relation to its preferred embodiment, it is to be understood that many other possible modifications and variations can be made without departing from the spirit and scope of the invention as hereinafter claimed.

What is claimed is:

1. A method of implementing a decentralized user-extensible system for storing and managing unified medical files comprising the steps of:
(A) providing at least one computing engine, at least one database, and at least one user interface managed by at least one remote server, wherein at least one personal computing (PC) device is communicably coupled with the remote server;
(B) receiving input from an administrator account through the user interface with the remote server in order to define a plurality of web controls and a plurality of webform templates, wherein each web control comprises a plurality of web-control metadata attributes, wherein each webform template comprises a plurality of webform metadata attributes, and wherein each webform template is associated with at least one web control from the plurality of web controls;
(C) storing the web controls and the webform templates in the database with the remote server;
(D) receiving a request from an end user account with the remote server to retrieve at least one specific webform template from the plurality of webform templates through the remote server;
(E) processing the specific webform template with the computing engine in order to produce a rendered webform from the specific webform template, wherein the rendered webform comprises the at least one web control of the specific webform template;
(F) displaying the rendered webform on the user interface to the end user account through the PC device;
(G) receiving at least one patient data value through the at least one web control of the rendered webform from the end user account through the user interface, wherein each patient data value corresponds to one of the at least one web control of the rendered webform;
(H) compiling the specific webform template, each patient data value, and a plurality of runtime-filled webform values with the computing engine in order to produce a filled webform;
(I) appending at least one runtime-filled data item value to each patient data value with the computing engine; and
(J) storing each patient data value of the filled webform as a filled data item in the database.

2. The method of implementing a decentralized user-extensible system for storing and managing unified medical files as claimed in claim 1 comprising the steps of:
providing that the at least one database comprises a template database and a production database;
storing the web controls and the webform templates in the template database with the remote server; and
storing each patient data value of the filled webform as a filled data item in the production database.

3. The method of implementing a decentralized user-extensible system for storing and managing unified medical files as claimed in claim 1 comprising the steps of:
providing that the at least one database comprises a template database and a production database;
receiving a report request through the PC device;
searching the template database to find a relevant webform template, wherein the relevant webform template corresponds to the report request;
searching the production database to find at least one relevant filled data item, wherein each relevant filled data item corresponds to the relevant webform template;
rendering each relevant filled data item with the relevant webform template as a rendered report with the computing engine; and
displaying the rendered report on the PC device.

4. The method of implementing a decentralized user-extensible system for storing and managing unified medical files as claimed in claim 1 comprising the steps of:
providing that the end user account is associated with a set of user permissions in the database;
providing that the specific webform template comprises a set of transaction rules;
comparing the set of transaction rules with the set of user permissions in order to validate the end user account as permitted to access the specific webform template;
retrieving the specific webform template from the database, if the user account is validated as permitted to access the specific webform template; and
displaying the specific webform template to the end user account through the user interface.

5. The method of implementing a decentralized user-extensible system for storing and managing unified medical files as claimed in claim 4 comprising the steps of:
providing each web control from the plurality of web controls being associated with at least one permission attribute;
comparing the permission attributes of each web control of the specific webform template to the set of user permissions of the end user account in order to identify at least one compatible control from the at least one control of the specific webform template;
displaying the at least one compatible control on the specific webform template to the end user account;

6. The method of implementing a decentralized user-extensible system for storing and managing unified medical files as claimed in claim 1 comprising the steps of:
providing that the at least one computing engine comprises a unified medical file (UMF) engine, wherein the UMF engine manages and processes data values received through the web controls;
compiling the specific webform template, each patient data value, and a plurality of runtime-filled webform values with the UMF engine in order to produce the filled webform; and
appending at least one runtime-filled data item value to each patient data value with the UMF engine.

7. The method of implementing a decentralized user-extensible system for storing and managing unified medical files as claimed in claim 1, wherein each of the plurality of web controls is associated with a specified medical identifier code.

8. The method of implementing a decentralized user-extensible system for storing and managing unified medical files as claimed in claim 1 comprising the steps of:
providing a patient computing device;
providing that the at least one database comprises a patient data repository, wherein the patient data repository is stored on the patient computing device;
storing each patient data value of the filled webform as a filled data item in the patient data repository, wherein each patient data value is associated with a patient account;
compiling the patient data values of the patient account into a patient electronic medical record (EMR) associated with the patient account with a unified medical file (UMF) engine from the at least one computing engine, wherein a set of user permissions associated with the patient account defines the patient account as a primary administrative entity for the patient EMR; and
storing the patient EMR in the patient data repository.

9. The method of implementing a decentralized user-extensible system for storing and managing unified medical files as claimed in claim 8 comprising the steps of:
receiving a request from the end user account with the remote server to display the patient EMR;
retrieving the patient EMR from the data repository with the remote server; and
displaying the patient EMR through the PC device.

10. The method of implementing a decentralized user-extensible system for storing and managing unified medical files as claimed in claim 8 comprising the steps of:
receiving a request with the remote server to access the patient EMR; and
sending an access request notification to the patient account.

11. The method of implementing a decentralized user-extensible system for storing and managing unified medical files as claimed in claim 1 comprising the steps of:
providing a patient computing device, wherein the patient computing device is communicably coupled with the remote server;
providing that the at least one database comprises a patient data repository, wherein the patient data repository is stored on the patient computing device;
providing a healthcare facility data repository, wherein the healthcare facility data repository is communicably coupled with the remote server;
concurrently storing a first copy of the patient electronic medical record (EMR) in the database, a second copy of the patient EMR in the patient data repository, and a third copy of the patient EMR in the healthcare facility data repository;
receiving user input to produce a modified patient EMR from the patient EMR; and
updating the first copy, the second copy, and the third copy of the patient EMR as the modified patient EMR.

12. The method of implementing a decentralized user-extensible system for storing and managing unified medical files as claimed in claim 12 comprising the steps of:
receiving user input from a patient account with the remote server to de-authorize the patient EMR;
deleting the first copy, and the second copy, and the third copy of the patient EMR through the patient computing device, the remote server, and the healthcare facility data repository, if the user input to de-authorize the patient EMR is received with the remote server.

13. The method of implementing a decentralized user-extensible system for storing and managing unified medical files as claimed in claim 1 comprising the steps of:
receiving input from the administrator account through the user interface to define at least one activity template through the UMF engine, wherein each activity template comprises at least one webform template, a webform template sequence, and at least one classification identifier;
receiving input through the user interface to instantiate a specific activity template from the at least one activity template in association with a patient user account;
retrieving and instantiating an invoice template from the database with the remote server along with the specific activity template, wherein the invoice template corresponds to the classification identifier of the specific activity template;
receiving user activity input through the user interface to progress through the specific activity template and the invoice template according to the webform template sequence of the specific activity template;
receiving user input through the user interface to finalize the specific activity template;
compiling the specific activity template, the invoice template, and the user activity input into an activity summary; and
storing the activity summary in the database and associating the activity summary with the patient user account.
